(19) United States
(12) Patent Application Publication
(10) Pub. No.: US 2015/0132746 A1
(43) Pub. Date: May 14, 2015

(54) SAMPLING MEDIUM

(71) Applicant: UNIVERSITY OF OSLO, Oslo (NO)

(72) Inventors: Astrid Gjelstad, Oslo (NO); Lars Erik Eng Eibak, Oslo (NO); Anne Bee Hegge, Oslo (NO); Knut Einar Rasmussen, Eiksmarka (NO); Stig Pedersen-Bjergaard, Oslo (NO)

(21) Appl. No.: 14/402,810

(22) PCT Filed: May 29, 2013
(86) PCT No.: PCT/IB2013/001553

§ 371 Date: Nov. 21, 2014

Related U.S. Application Data
(60) Provisional application No. 61/653,673, filed on May 31, 2012.

(51) Int. Cl. G01N 1/36; G01N 33/94

(52) U.S. Cl. CPC G01N 1/36; G01N 33/9486; G01N 33/946; G01N 2001/364
USPC 435/6.1; 436/174; 436/92; 435/309.1; 436/112; 436/111; 436/501; 436/86; 436/71; 436/94; 435/7.1; 435/29; 435/30; 422/551

(57) ABSTRACT

Provided herein is technology relating to the collection of biological samples and particularly, but not exclusively, to compositions, methods, and uses related to using a biopolymer substrate to collect biological samples for analysis.

### FIELD OF INVENTION

Provided herein is technology relating to the collection of biological samples and particularly, but not exclusively, to compositions, methods, and uses related to using a biopolymer substrate to collect biological samples for analysis.

### BACKGROUND

Numerous clinical analyses are based on testing biological fluids such as, e.g., blood, saliva, semen, sweat, etc. For example, conventional drug analysis from human specimens has traditionally been conducted using whole blood or urine. However, there is an increasing interest in the use of oral fluid to investigate driving under the influence, to monitor drug therapy, and for clinical toxicology and criminal justice applications. A sampling procedure based on oral fluid collection is less invasive compared to conventional blood and urine collection (Choo R E, Huestis M A. Oral fluid as a diagnostic tool. Clinical Chemistry and Laboratory Medicine 2004; 42:1273-87; Gallardo E, Queiroz J A. The role of alternative specimens in toxicological analysis. Biomedical Chromatography 2008; 22:795-821); also, the infection risk is lower (Langel K, et al. Drug testing in oral fluid—evaluation of sample collection devices. J Anal Toxicol 2008; 32:393-401). Oral fluid collection is less susceptible to adulteration and substitution, which is a major concern for urine sample collection (Gallardo, supra). Applications based on oral fluid collection for determination of drug use have been demonstrated by the RoadSIde Testing Assessment (ROSITA) and several workplace surveys of drug abuse (Bosker W M, Huestis M A. Oral fluid testing for drugs of abuse. Clinical Chemistry 2009; 551910-31).

A healthy individual produces 500-1500 mL of oral fluid daily; however, the oral fluid secretion is highly sensitive to physiological stimuli and consequently fluctuates throughout the day (Aps J K M, Martens L C. The physiology of saliva and transfer of drugs into saliva. Forensic Sci Int 2005; 150:119-31). A typical oral fluid sampling volume is 50-500 μl. Human oral fluid has a low protein content (˜3 g/L) compared to human plasma (˜70 g/L), and thus provides a less complex matrix for subsequent analysis (Aps, supra). Conventional oral fluid collection involves sampling in the oral cavity with a cellulose pad; this sampling pad is transferred into a storage diluent and is then transported to the analytical laboratory for sample preparation and analysis. The purpose of the diluent is primarily to preserve (e.g., to prevent microbial growth) after sampling and during storage (Drummer O H. Drug testing in oral fluid. Clin Biochem Rev 2006; 27:147-59).

For collecting oral fluids, several collection kits are marketed for sample collection based on sampling the oral cavity with a cellulose pad followed by storage of the pad in a preservation fluid. The analytical procedure involves extraction of analytes from the preservation fluid followed by LC-MS/MS. However, the conventional technologies present difficulties for analysis related to the adsorption of analytes to the sampling pad and ion suppression in the MS by components of the preservation fluid. For example, MS matrix effects due to constituents in the sampling pad and the diluent have been reported as a major drawback with conventional oral fluid collection devices. This ion-suppression or enhancement is mainly ascribed to the surfactants and preservatives present in the diluents mixed with the sampling pad (Bosker, supra). To isolate the analytes selectively from the sample and eliminate the matrix effect, an extensive sample preparation procedure is required prior to analysis by liquid chromatography tandem mass spectrometry 94 (LC-MS-MS) (Annesley T M. Ion suppression in mass spectrometry. Clinical Chemistry 2003; 49:1041-4; Matuszewski B K, et al. Strategies for the assessment of matrix effect in quantitative bioanalytical methods based on hplc-ms/ms. Anal Chem 2003; 75:3019-30). In addition to increasing analysis time, these problems often lead to unreliable results.

While oral fluid is used when possible because oral sampling is less invasive compared to conventional blood and urine collection, some analyses will continue to be based on blood testing. For blood testing, dried blood spots are becoming the primary sampling method for studies conducted in industrial and hospital laboratories, e.g., for clinical diagnostics and for pharmacokinetic and for toxicokinetic studies.

The analysis of small drug substances in whole blood samples collected and stored on filter paper, known as dried blood spots (DBS), has increased rapidly the last years. The major interest in switching from traditional wet plasma to DBS is due to the ethical, economical, and practical advantages (Keevil B G. The analysis of dried blood spot samples using liquid chromatography tandem mass spectrometry. Clinical Biochemistry 2011; 44:110-8; Timmerman P, et al. Ebf and dried blood spots: From recommendations to potential resolution. Bioanalysis 2011; 3:1787-9). Conventional methods are based on isolation of plasma from whole blood followed by centrifugation. Thereafter, plasma samples are prepared for analysis using protein precipitation, solid-phase extraction, or liquid-liquid extraction. This time-consuming process limits the number of samples that can be tested. The small blood volumes required for DBS (less than 100 μl) makes this approach particularly suitable for quantification of drug substances in drug metabolism (DM), pharmacokinetic (PK), and toxicokinetic (TK) studies. In addition, it offers the advantage of less invasive sampling (e.g., finger or heel prick) instead of sampling with the conventional cannula, which is a great advantage in pediatric studies and in therapeutic drug monitoring (TDM) (Merton G, et al. Accuracy of cyclosporin measurements made in capillary blood samples obtained by skin puncture. Therapeutic Drug Monitoring 2000; 22:594-8; Woods K, et al. Patient preferences for capillary vs. Venous in determination in an anticoagulation clinic: A randomized controlled trial. Thrombosis Research 2004; 114:161-5). In addition, easy storage at room temperature and easy shipment to analytical laboratories offer further advantages (Mei J V, et al. Use of filter paper for the collection and analysis of human whole blood specimens. Journal of Nutrition 2001; 131:1631S-6S). The stability of DBS under different storage conditions has been evaluated and considered as satisfactory (Strnadová K A, et al. Long-term stability of amino acids and acylcarnitines in dried blood spots. Clinical Chemistry 2007; 53:717-22; Alfazil A A, Anderson R A. Stability of benzodiazepines and cocaine in blood spots stored on filter paper. J Anal Toxicol 2008; 32:511-5; Adam B W, et al. The stability of markers in dried-blood spots for recommended newborn screening disorders in the united states. Clinical Biochemistry 2011; 44:1445-50). Patients can administer the finger prick at home without the need of trained phlebotomists.

DBS cards are now available from several manufacturers. Most cards are made of pure cellulose and can include substances that lyse cells and denature proteins on contact (Edelbroek P M, et al. Dried blood spot methods in therapeutic drug monitoring: Methods, assays, and pitfalls. Therapeutic Drug Monitoring 2009; 31:327-36). Recently, non-cellulose based cards (e.g., Agilent Bond Elute DMS) have become available as an alternative. Commercial cards have circles marked for blood spotting. Handling and analysis is simple and involves only a few steps. One method of sample collection is a finger or heel prick followed by blood flow into a glass capillary or microvessel. Typically, 10-20 μl of blood is applied for each spot by the capillary or by a micropipette and the blood spots are allowed to dry for a period of 2 hours at room temperature (Keevil, supra). For analysis, a circular portion of each dried blood spot or the whole blood spot is punched out into a vial. The analytes are eluted by a few hundred milliliters of an organic solvent, e.g., typically methanol or methanol-water mixtures containing an internal standard. The sensitivity and selectivity offered by liquid chromatography tandem mass spectrometry (LC-MS-MS) have contributed to the success of the technique and LC-MS-MS is the golden standard for DBS analysis (Keevil, supra).

Despite the many advantages offered by DBS analysis on commercial cards there are still some drawbacks. Elution of analytes is time consuming and elution times of 1 hour are common (Keevil, supra). Elution volumes of a few hundreds of microliters lead to a great reduction of assay sensitivity and enrichment by evaporation and reconstitution of the sample in a smaller volume may be necessary to improve sensitivity. Some constituents may contribute to ion suppression in MS and additional cleanup by an extraction method may be necessary.

### SUMMARY

These problems are minimised or eliminated by storing biological fluids as dried spots (e.g., DBS spots, DMS spots) on a sampling material made from a water-soluble biopolymer or a mixture of water-soluble polymers and using an extraction technique (e.g., electromembrane extraction (EME)) for enrichment and cleanup. As such, for many applications biopolymer spotting cards can replace insoluble cellulose- and non-cellulose-based spotting cards. In particular, the time needed to prepare a sample using an alginate or chitosan spotting card is shortened considerably because these biopolymers dissolve in less than five minutes in aqueous acidic solutions. In addition, higher analyte recoveries are obtained from biopolymers compared to conventional spotting cards. Biopolymer spotting cards significantly increase the number of samples that can be analysed because of shorter sample preparation times. As described herein, the technology provides for the safe storage of biological fluids. Data collected using samples obtained on biopolymer spotting cards validated the use of the biopolymer in subsequent analytical procedures. The invention broadens the usage of soluble biopolymers as sampling media for biological fluids.

The technology provided herein is related in one aspect to sampling oral fluids as dried drops on alginate and chitosan foam. For example, during the development of embodiments of the technology, experiments were conducted in which freshly prepared oral fluid was spiked with model analytes and collected on alginate or chitosan foam. The samples were subsequently dissolved in dilute HCl, the model analytes were isolated from the sample into an acidic acceptor solution with an optimized EME setup, and the analytes were analyzed with LC-MS. In some experiments, the method produced extraction recoveries in the range of 25-82% from oral fluid spiked with buprenorphine, methadone, methamphetamine, para-methoxyamphetamine, and para-methoxymethamphetamine in 5 minutes of extraction. Linearity was examined in the range 25-1000 ng/ml for all 5 model analytes stored on both alginate and chitosan foams. The correlation coefficients were above 0.9885 for all model analytes. The reported RSD values were below 15% for low concentrations (50 ng/ml) and below 20% for high concentrations (1000 ng/ml). Long-term stability was examined by storing oral fluid spiked with model analytes on alginate and chitosan foams for 30 days under relevant storage conditions. The stability within 30 days of storage was considered satisfactory. A post-column infusion experiment indicated no matrix effect and demonstrated that EME provides efficient sample cleanup from complex biological matrices in 5 minutes. The proposed method evaluation in terms of the FDA's guidance for industry concerning bioanalytical method validation was considered as acceptable.

Alginate and chitosan foams were tested as sampling media for the analyses of blood. For example, during the development of embodiments of the technology, samples of whole blood containing citalopram, loperamide, methadone, and sertraline as model substances were spotted on alginate and chitosan foams. After drying and room temperature storage, punched out dried blood spots and the biopolymer were dissolved in dilute HCl. With alginate as sampling medium, the analytes dissolved completely after 3 minutes. Enrichment and cleanup were performed by electromembrane extraction. The analytes were collected in formic acid as an acceptor phase and the extracts were analyzed by LC-MS. The recoveries were comparable to recoveries obtained after extraction of the model substances from wet plasma.

Sample preparation of blood spots on conventional cards (Whatman FTA DMPK and Agilent 47 Bond Elute DMS) followed elution procedures recommended by the manufacturers. The recoveries from the conventional media for most of the model analytes were lower than the recoveries obtained from biopolymers as sampling media. The procedure used for Agilent Bond Elute DMS showed higher recoveries than the procedure used for Whatman FTA DMPK-A, but the time needed for sample preparation was significantly longer (nearly 2 hours). The stability of the model substances on the alginate foam was investigated under different storage conditions and was acceptable within 50 days of storage. The limit of quantification (LOQ) was 1.2, 5.5, 2.0, and 5.3 ng/ml for citalopram, loperamide, methadone, and sertraline, respectively. Linear calibration graphs were obtained in the range 17.5-560 ng/ml with r² values of 0.983-0.995 and the relative standard deviations were below 20%. Post column infusion experiments indicated that ion suppression was non-existent with the EME setup described herein.

Accordingly, the technology provided herein relates to, in one aspect, articles or compositions comprising a biopolymer spotting card and a biological fluid. While the technology is not limited in the biopolymer from which the spotting card is made, in some embodiments, the biopolymer is alginate or chitosan. Furthermore, the technology is not limited in the biological fluid that is sampled using the biopolymer spotting card. For example, in some embodiments, the biological fluid is blood, a blood fraction, or a component of blood; in some embodiments, the biological fluid is oral fluid; and in some embodiments, the biological fluid is sputum, semen, sweat, urine, cerebrospinal fluid, tears, saliva, breast milk, or vaginal fluid.

In some embodiments, the biopolymer or the combination of biopolymers is dissolvable in a dilute acid; as such, in some embodiments, the compositions further comprise a dilute acid. In some embodiments, a biological fluid is sampled to obtain an analyte. Thus, in some embodiments, the biological fluid comprises an analyte such as, e.g., a drug, toxin, metabolite, nucleic acid, protein, lipid, or therapeutic agent. In some embodiments, the biopolymer card changes color when exposed to a sample to indicate the location of the sample on the card. In some embodiments, therefore, the compositions (e.g., a biopolymer card) comprise a dye that is a visible color when contacted with the biological fluid. In some embodiments, an excipient is added to the biopolymer to modify its solubility. In some embodiments, the biopolymer is semi-synthetic, e.g., chitosan. The compositions are not limited in their form, shape, or size. For example, in some embodiments the biopolymer is in the form of a porous sheet of foam.

The biopolymer card is used, in some embodiments, to obtain one or more samples. Consequently, some embodiments of the technology provide an array of spots on a biopolymer spotting card wherein a spot comprises a biological fluid. The spots comprise, in some embodiments, one of the compositions as described above.

In another aspect, the technology provided herein relates to methods for sampling a biological fluid. For instance, in some embodiments, the technology provides a method comprising spotting the biological fluid on a biopolymer spotting card; and drying the biological fluid on the biopolymer spotting card. Some embodiments provide further steps such as, e.g., storing the biological fluid on the biopolymer spotting card (e.g., for a period of time less than or equal to 50 days at room temperature). In related embodiments, the technology provides a method of measuring an analyte, the method comprising spotting a biological fluid on a biopolymer spotting card; dissolving the biopolymer spotting card; recovering the analyte; and measuring a property of the analyte. Some embodiments comprise additional steps such as, e.g., cutting the biopolymer spotting card; measuring a property of the analyte using mass spectrometry; recovering the analyte using electromembrane extraction; dissolving the biopolymer using a dilute acid (e.g., such as hydrochloric acid); and storing the biological fluid on the biopolymer spotting card.

In some embodiments, the technology provides a use of a biopolymer card for sampling a biological fluid. In some embodiments, the biopolymer is alginate and in some embodiments the biopolymer is chitosan. The technology includes, for example, use of a biopolymer card for sampling a biological fluid such as blood, oral fluid, sputum, semen, sweat, urine, cerebrospinal fluid, tears, saliva, breast milk, and/or vaginal fluid. In some embodiments, the technology relates to use of a biopolymer card to store a biological fluid. The biopolymer is, in some embodiments, alginate or chitosan. The biological fluid is, in some embodiments, blood, oral fluid, sputum, semen, sweat, urine, cerebrospinal fluid, tears, saliva, breast milk, and/or vaginal fluid. In some embodiments, the use comprises storing the biological fluid at room temperature for up to 50 days.

In another aspect of the technology, embodiments relate to an analytical sample comprising an analyte, obtainable by a method comprising the steps of spotting a biological fluid on a biopolymer spotting card; dissolving the biopolymer spotting card; and recovering the analyte. In some embodiments, the recovering comprises using electromembrane extraction and in some embodiments, the dissolving comprises using a dilute acid.

Moreover, the technology provide embodiments of a biopolymer card for use as a sampling medium, and a biopolymer card for use as a sampling medium to analyse a biological fluid. Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a drawing of an electromembrane extraction apparatus. FIG. 1a shows one embodiment and FIG. 1b shows another embodiment.

FIG. 2 shows a chromatogram obtained after EME from oral fluid stored on alginate foam.

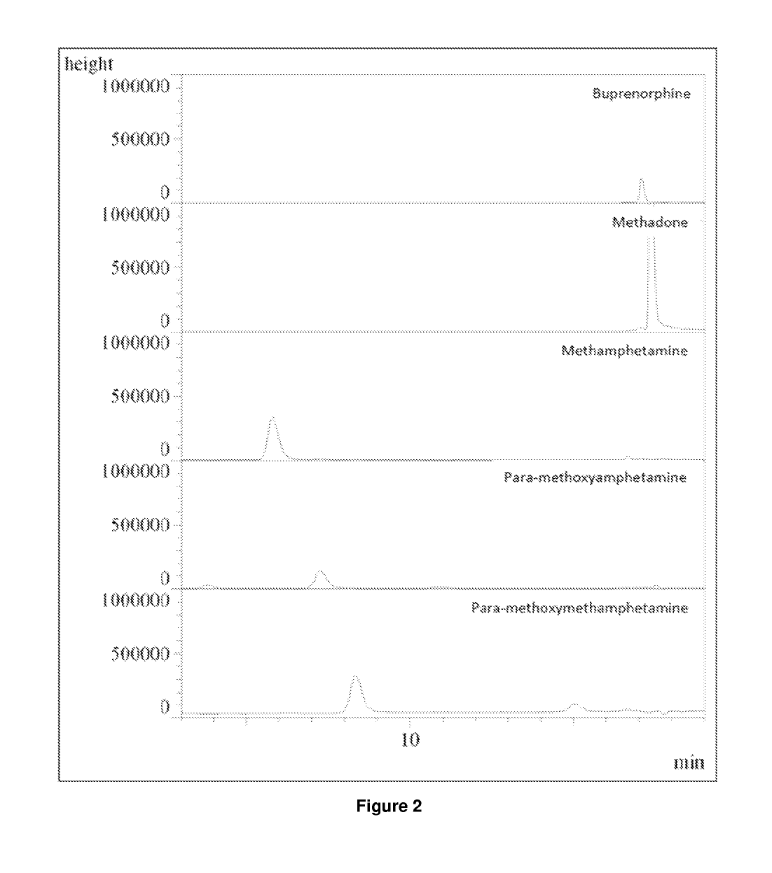

FIG. 3 shows a post column chromatogram obtained from blank saliva stored on alginate.

FIG. 4 shows a post column chromatogram obtained from blank saliva stored on chitosan.

FIG. 5 shows chromatograms obtained for citalopram (m/z 325) using Bond Elute DMS (a), FTA DMPK-A (b), alginate-EME (c), and alginate-precipitation (d).

FIG. 6 shows in (a) an MS chromatogram obtained with a standard solution of citalopram (1), Lu 10-202 (IS) (2), methadone (3), loperamide (4), and sertraline (5) having a concentration of 500 ng/ml. FIG. 5 (b) is an MS chromatogram obtained after EME extraction of blank DBS on alginate foam with post column infusion of model substances and internal standard with a concentration of 250 ng/ml.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to the collection of biological samples and particularly, but not exclusively, to compositions, methods, and uses related to using a biopolymer substrate to collect biological samples for analysis.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the

### DETAILED DESCRIPTION

Throughout the specification and claims, the following terms take the meanings explicitly associated herein, unless the context clearly dictates otherwise.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

### Embodiments of the Technology

### 1. Biopolymers

Biopolymers are polymers produced by living organisms and/or made from biological materials that are found in nature, derived from materials found in nature, and/or modified forms of materials found in nature. Biopolymers may be natural, synthetic, or semi-synthetic (e.g., comprising both naturally occurring and synthetic components). Since they are polymers, biopolymers contain monomeric units that are covalently bonded to form larger structures. There are three main classes of biopolymers based on the differing monomeric units used and the structure of the biopolymer formed: polynucleotides (e.g., polymers composed nucleotide monomers; polypeptides (e.g., short polymers of amino acids); and polysaccharides (e.g., linear bonded polymeric carbohydrate structures). As an example, cellulose is the most common organic compound and biopolymer on Earth. About 33 percent of all plant matter is cellulose. The cellulose content of cotton is 90 percent and that of wood is 50 percent.

The technology provided herein relates to the use of sugar-based biopolymers (e.g., polysaccharides). These can be classified as anionic, cationic, and non-ionic. Alginate is an example on an anionic polymer, chitosan is an example of a cationic polymer, and carboxymethylcellulose (CMC) is an example of a non-ionic biopolymer. Other sugar-based soluble biopolymers contemplated by the technology include, e.g., water soluble cellulose derivatives, chitin, dextran, pullulan, hyaluronic acid and salts thereof, pectins, carrageenans, and xanthan. In some embodiments, the biopolymer comprises a combination or mixture of one or more biopolymers, and in some embodiments other components (e.g., an excipient) are added to modify a physical characteristic (e.g., solubility, rate of dissolution, strength, etc.) of the biopolymer, e.g., in a biopolymer formulation.

Alginate and chitosan are water-soluble biopolymers made up of repeating monomers. Alginate is the salt of alginic acid and is an anionic polysaccharide distributed widely in the cell walls of brown alga. It is an anionic polysaccharide consisting of homopolymeric blocks of (1-4)-linked β-D-mannuronate and the C-5 epimer α-L-guluronate. The aqueous solubility of alginate decreases at pH below about 3 due to protonation of the mannuronic and guluronic acid groups (e.g., pKₐ values are 3.65 and 3.38 for guluronic and mannuronic acid, respectively) (A. Haug, Larsen B. The solubility of alginate at low ph Acta chemica Scandinavica 1963; 21:1653).

Chitosan is a cationic polysaccharide containing more than 5000 glucosamine units and is obtained commercially from shrimp and crab shell chitin. The pKa value for glucosamine is 6.3 and the reported pH values for complete solubility are in the lower pH range (V\dot{a}rum K M, et al. Water-solubility of partially n-acetylated chitosans as a function of ph: Effect of chemical composition and depolymerisation. Carbohydrate Polymers 1994; 25:65-70). See also, Yi H, et al. Biofabrication with chitosan. Biomacromolecules 2005; 6:2881-94.

Alginate and chitosan exhibit biocompatibility and can be manufactured in many forms, e.g., as fibers, foams, or gels. They are manufactured commercially for extant applications such as wound management, tissue engineering, and controlled drug release. These biopolymers are water soluble and generally insoluble in most other solvents including alcohol. Sheets of foam with a porous paper-like structure are prepared and serve as a sampling medium. Among the physical foam properties that can be controlled are pore size, foam thickness, tensile strength, rate of disintegration in water, and density.

### 1. Extraction of Analytes

As the solution contains the dissolved biopolymer in addition to biological (e.g., blood) components and the analytes, extraction of analytes or precipitation of the biopolymer is in some cases necessary prior to analysis. Therefore, the analytical protocol includes sample preparation, separation, and detection of the analytes in the final extract.

An example of an extraction technique is electromembrane extraction. In electromembrane extraction (EME) (Pedersen-Bjergaard S, Rasmussen K E. Electrokinetic migration across artificial liquid membranes—new concept for rapid sample preparation of biological fluids. J Chromatogr A 2006; 1109:183-90), charged substances migrate through a supported liquid membrane (SLM) in response to an electro-kinetic field (Balchen M, et al. Electrokinetic migration of acidic drugs across a supported liquid membrane. J Chromatogr A 2007; 1152:220-5; Balchen M, et al. Rapid isolation of angiotensin peptides from plasma by electromembrane extraction. J Chromatogr A 2009; 1216:6900-5; Jamt R E G, et al. Electromembrane extraction of stimulating drugs from undiluted whole blood. J Chromatogr A 2012; 1232:27-36; Payán M R, et al. Electromembrane extraction (eme) and hplc determination of non-steroidal anti-inflammatory drugs (nsaids) in wastewater samples. Talanta 2011; 85:394-9.). For example, the method effects the selective extraction of basic or acidic substances from water, plasma, urine, whole blood, breast milk, and tap water (Kjelsen I J O, et al. Low-voltage electromembrane extraction of basic drugs from biological samples. J Chromatogr A 2008; 1180:1-9; Eskandari M, et al. Microextraction of mebendazole across supported liquid membrane forced by ph gradient and electrical field. J Pharmaceut Biomed 2011; 54:1173-9; Jamt R E G, et al. Electromembrane extraction of stimulating drugs from undiluted whole blood. J Chromatogr A 2011; Seidi S, et al. Determination of thebaine in water samples, biological fluids, poppy capsule, and narcotic drugs, using electromembrane extraction followed by high-performance liquid chromatography analysis. Anal Chim Act 2011; 701:181-8).

Under the applied electrical field, substances migrate from a sample solution through the SLM and toward the electrode with opposite charge located in the lumen of the hollow fiber. The aqueous extract produced by EME is directly compatible with LC-MS (Pedersen-Bjergaard, supra). The SLM serves as a sample cleanup barrier, excludes the matrix constituents, and consequently limits the matrix effect in the MS. EME has been used, e.g., to clean up post-mortem whole blood samples comprising six basic drugs of abuse within 5 minutes by utilizing a 15 V battery (Jamt, supra). EME has also been applied to process oral fluid (Seidi S, et al. Electromembrane extraction of levamisole from human biological fluids. J Sep Sci 2011; 34:585-93).

EME extractions from 500 to 1000 μl samples are completed within 10 minutes of extraction time at 10-300 V (Gjelstad A, et al. Electromembrane extraction: A new technique for accelerating bioanalytical sample preparation. Bioanalysis 2011; 3:787-97). To simplify the EME setup and reduce the required amount of chemicals and sample, in some embodiments the adjustable power supply is replaced with a 9 V battery to extract analytes from 70 μl untreated whole blood and human plasma (Eibak L E E, et al. Kinetic electro membrane extraction under stagnant conditions—fast isolation of drugs from untreated human plasma. J Chromatogr A 2010; 1217:5050-6). Another substantial benefit is the enrichment factor obtained without any need for evaporation or reconstitution. The aqueous extracts provided with EME are directly injected into the LC-MS-MS instrument. For example, exhaustive electromembrane extraction of basic drugs from undiluted human plasma can be followed by LC-MS (Eibak L E E, et al. Exhaustive electromembrane extraction of some basic drugs from human plasma followed by liquid chromatography-mass spectrometry. J Pharmaceut Biomed 2012; 57:33-8). In the development of some embodiments of the technology provided herein, this approach was used to extract basic model substances from an acidic aqueous solution of a dissolved blood spot and the biopolymer.

The technology is not limited to EME and contemplates other methods of isolation an analyte from the biopolymer. For example, precipitation of the biopolymer with acetonitrile is an alternative sample preparation technique (e.g., as described below). Also contemplated are sample preparation techniques comprising liquid-liquid extraction and solid-phase extraction, and combinations of these sample preparation techniques.

Moreover, the separation and detection techniques are not be limited to LC-MS. Also contemplated are other detection techniques, e.g., as combined with LC, such as UV detection, fluorescence detection, and electrochemical detection. Also, it is contemplated that the technology comprises capillary electrophoresis combined with UV and/or fluorescence detection; and gas chromatography coupled to detectors such as mass spectrometry, FID, and nitrogen-phosphor detection. Immunological methods are also contemplated in embodiments of the technology.

### 2. Dried Matrix Spot Sampling

Dried matrix spot (DMS) sampling involves storing a low microliter volume of a biological sample spotted on paper-like structures (Keevil B G. The analysis of dried blood spot samples using liquid chromatography tandem mass spectrometry. Clinical Biochemistry 2011; 44:110-8). DMS provides dry storage of the sample and thus limits enzymatic and hydrolytic degradation. DMS sampling from, e.g., whole blood has been used in applications such as drug metabolism (DM), and in pharmacokinetic (PK) and toxicokinetic (TK) studies. For colourless fluids like plasma, cerebrospinal fluid (CSF), and urine, applications use a color indicating card (e.g., as supplied by Whatman) that identifies the location of the occupied area (“spot”) by a color change.

Sampling by DMS offers an easier and less invasive sampling procedure compared to conventional whole blood collection with a cannula; the sampling volume is substantially reduced and the sample can be stored at room temperature in sealed plastic bags (Keevil, supra). A single blood drop (10-20 μl) provided by a finger- or heel prick is considered sufficient to quantify endogenous drug levels.

The advantages provided by DMS, e.g., low sample volume and dry storage of samples, addresses some challenges associated with conventional oral fluid collection procedures. For example, one of the most frequently reported effects of drug use is reduced oral fluid production (Aps J K M, Martens L C. The physiology of saliva and transfer of drugs into saliva. Forensic Sci Int 2005; 150:119-31; Chicharro J L, et al. Saliva composition and exercise. Sports Medicine 1998; 26:17-27; Walsh N P, et al. Saliva parameters as potential indices of hydration status during acute dehydration. Medicine and Science in Sports and Exercise 2004; 36:1535-42). In such cases, the sampling process can be highly demanding, e.g., because of the smaller sample volume available for collection. DMS provides a technology for collection and preservation of small samples. These samples are appropriate for LC-MS-MS analysis, which is highly sensitive and can quantify endogenous drug levels from these types of low microliter volumes of oral fluid. In addition, the storage of oral fluid as spots on a material circumvents the need for preservation with a diluent, some of which are proprietary compositions.

Although the disclosure herein refers to certain illustrated embodiments, it is to be understood that these embodiments are presented by way of example and not by way of limitation.

### EXAMPLES

During the development of embodiments of the technology provided herein, experiments were performed to investigate the storage of biological fluids (e.g., oral fluid and blood) as dried spots on alginate and chitosan foams followed by EME to isolate basic model analytes selectively from the biological fluids. The properties of these biopolymers as spotting cards were compared with the properties of conventional cellulose spotting cards (Whatman FTA DMPK-A) and non-cellulose spotting cards (Agilent Bond Elute DMS).

In brief, after sampling oral fluid by spotting it on the sampling medium of interest, the spot was dried under room temperature for 3 hours and subsequently punched out. The punched-out oral fluid spot was dissolved in 300 μl of 1 mM HCl for 5 minutes, providing a translucent solution comprising the analytes, oral fluid, and biopolymer. The analytes were selectively isolated by EME within 5 minutes of extraction. The time needed for each sample including dissolution, isolation, and enrichment is 10 minutes. The aqueous extracts obtained were directly compatible with LC-MS analysis and a series of experiments indicated no ion suppression or enhancement in the final extract. The analytes are also stable within 30 days under relevant storage conditions.

For blood samples, the biopolymer and the dried blood spot was dissolved in 3 minutes in 1 mM HCl. Basic drugs as model substances were analyzed by LC-MS after EME and after precipitation of the biopolymer with ice-cold acetonitrile. EME fully demonstrated its sample cleanup properties and extracted the model substances selectively and exhaustively from the dissolved DBS. There was no indication of ion suppression in LC-MS. Total sample preparation times were 15 minutes including dissolution and extraction. Biopolymers as spotting cards offers considerable saving in time compared to commercial spotting cards and the recoveries are generally higher. The stability of the model drug was considered satisfactory after 50 days of storage.

### Materials and Methods

### Chemicals

Methadone hydrochloride, 1-isopropyl nitrobenzene (IPNB), tris(2-ethylhexyl)phosphate (TEHP), loperamide hydrochloride, and tri fluoracetic acid (TFA) were all obtained from Sigma-Aldrich (St. Louis, Mo.). Methamphetamine was from Lipomed GmbH (Weil am Rhein, Germany) and buprenorphine hydrochloride was from NMD (Oslo, Norway). Para-methoxyamphetamine and para-methoxymethamphetamine was provided by Norwegian institute of public health (NIPH). Water was produced by a Milli-Q water purification system (Molsheim, France). Formic acid, methanol, and acetonitrile were from Merck (Darmstadt, Germany). Drug-free human oral fluid was obtained from a healthy volunteer at the School of Pharmacy (University of Oslo, Norway) and stored at −32° C.

### Standard Solutions

A stock solution of buprenorphine, methadone, methamphetamine, para-methoxyamphetamine, and para-methoxymethamphetamine with a concentration of 1 mg/ml was prepared in ethanol. Calibration standards at 25, 50, 200, 500, 750, and 1000 ng/ml were prepared by diluting the stock solution with fresh oral fluid. Subsequently, aliquots of 10 μl were spotted on the sampling medium of interest.

Methadone and loperamide were dissolved at 1 mg/ml to obtain appropriate standard solutions of methadone and loperamide. To obtain standard solutions at 1 mg/ml of sertraline and citalopram, a sertraline hydrochloride 50 mg tablet from Pfizer Italiana (Latina, Italy) was extracted with 50 ml ethanol and a citalopram hydrobromide 20 mg tablet from H. Lundbeck (Copenhagen, Denmark) was extracted with 20 ml ethanol. The internal standard Lu 10-202, a citalopram analogue (fluorine is replaced by chlorine in para position to the aromatic ring) was obtained from H. Lundbeck. 2-Nitrophenyl octylether was from Sigma-Aldrich (St. Louis, Mo.).

For blood studies, whole blood was evacuated and stored in BD (New Jersey) Vacutainer sodium heparin/lithium heparin tubes. Stock solutions of model substances with a concentration of 1 mg/ml was prepared in ethanol. Calibration standards at 17.5, 35, 70, 140, 280, and 560 ng/ml were prepared by diluting the stock solution with fresh whole blood. Subsequently aliquots were spotted on the sampling medium of interest.

### Sampling Medium

Alginate foam was provided in house and chitosan foam was produced at FMC BioPolymer AS/NovaMatrix (Sandvika, Norway). The conventional commercial cards examined were FTA DMPK-A cards produced by Whatman (Kent, United Kingdom) and Bond Elute DMS produced by Agilent (Santa Clara, Calif.).

Oral fluid sampling

A 10-μl volume of freshly prepared oral fluid spiked with model analytes was applied to the sampling medium of interest and dried at room temperature for 3 hours. After drying, the entire dried oral fluid spot was punched out (8 mm diameter) with a puncher. Subsequently, the dried oral fluid spot was dissolved in 300 μl of 1 mM HCl in a 08-CRV(A) vial (Chromacol, Trumbull, Conn.) with a total volume of 700 μl, an internal diameter of 7 mm, and height of 32 mm. The analytes were isolated from the solution by EME as described below.

### Blood Sampling

For testing the water soluble biopolymers, 10 μl freshly prepared whole blood spiked with model substances was applied to the sampling medium and dried at room temperature for 3 hours.

In dried blood matrix spotting, typically a small portion of the spot is punched out for analysis. This procedure works well when the blood spot is consistent and when the dispersion of analyte in the spot is homogeneous. The dried blood spot occupied a diameter of 5 mm. In the present investigation focus was on dissolution and extraction of analytes from the biopolymers and the whole blood spot was punched out for analysis using a homemade 8 mm puncher.

As such, after drying, the entire DBS was punched out (8 mm diameter) with a puncher and 2 μl internal standard was added and dried for another 30 minutes. Subsequently, the dried blood drop was dissolved in 300 μl of 1 mM hydrochloric acid in a 1500 μl vial with internal diameter of 10 mm and height of 32 mm (Agilent Technologies, Germany) with internal diameter of 10 mm and height of 32 mm. The solution was extracted by EME as described herein.

In an alternative procedure, the solution was added to 300 μl of ice-cold acetonitrile to precipitate the biopolymer. The solution was mixed for 5 min and centrifuged. The supernatant was evaporated to dryness and the residue was dissolved in 100 μl of mobile phase A and 20 μl was injected into LC-MS.

For testing the conventional Whatman FTA DMPK-A, 20 μl freshly prepared whole blood spiked with model substances was applied to the sampling medium and dried under room temperature for 3 hours. After drying, the DBS was punched out (3 mm diameter) with a puncher and subsequently eluted with 100 μl of 0.1% formic acid in 80% methanol for 10 minutes at 3000 rpm. The dissolution process was conducted in a 1500-μl vial with internal diameter of 10 mm and height of 32 mm. After elution the solution was centrifuged and 20 μl was injected into LC-MS.\

For testing the conventional Agilent Bond Elute DMS, 20 μl freshly prepared whole blood spiked with model substances was applied to the sampling medium and dried under room temperature for 3 hours. After drying, the DBS was punched out (3 mm diameter) with a puncher and subsequently dissolved in 300 μl of 0.1% formic acid in 80% methanol. The dissolution was conducted in a 1500-μl vial with internal diameter of 10 mm and height of 32 mm. After 60 minutes of elution at 3000 rpm the solution was centrifuged, evaporated to dryness, reconstituted in 200 μl of mobile phase A, and 20 μl was injected into LC-MS.

### Electromembrane Extraction (EME)

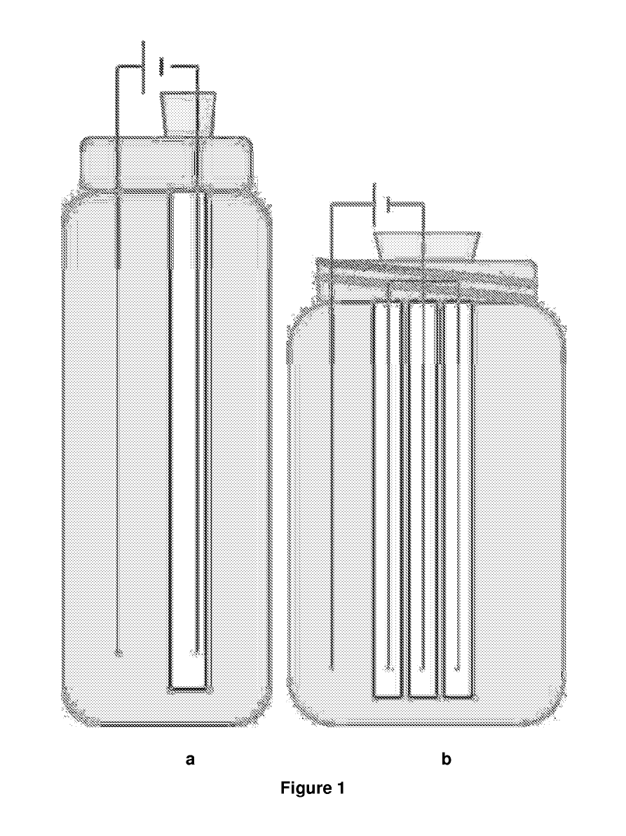

One embodiment of the EME apparatus is illustrated in FIG. 1a. A piece of PP Q3/2 polypropylene hollow fiber (Membrana, Wuppertal, Germany) with a pore size of 0.2 μm, a wall thickness of 200 μm, an inner diameter of 1.2 mm, and a length of 26 mm was closed at the lower end by mechanical pressure and the upper end was connected and sealed to a pipette tip (Finntip 200 Ext, Thermo 169 Scientific, Vantaa Finland). The supported liquid membrane was made by impregnating the pores of the hollow fiber for 5 seconds with an organic liquid consisting of 10% TEHP in IPNB. The excess organic phase was gently removed with a medical wipe. A 25-μl volume of 0.1% TFA was filled with an airtight syringe (Hamilton, Bonadus, Switzerland) in the lumen of the porous hollow fiber. Platinum wires with a diameter of 0.5 mm (K. A Rasmussen, Hamar, Norway) were connected to the power supply and utilized as electrodes. The anode was placed in the sample compartment and the cathode was placed in the lumen of the porous hollow fiber. A power supply ES 0300-0.45 from Delta Power Supplies (Delta Electronika, Zierikzee, The Netherlands) was operated at 25 V. The sample compartment was agitated at 900 rpm with an IKA MS 3 digital vortex mixer (Staufen, Germany). The total extraction time was set to 5 minutes. The extracts obtained with EME were diluted 1:1 with mobile phase A and 20 μl was injected into LC-MS.

In an alternative embodiment of the apparatus shown in FIG. 1b, a piece of PP Q3/2 polypropylene hollow fiber (Membrana, Wuppertal, 192 Germany) with pore size 0.2 μm, wall thickness 200 μm, inner diameter of 0.6 mm, and a length of 30 mm was closed in the lower end by mechanical pressure. The supported liquid membrane was made by impregnating the pores of three porous hollow fibers with 2-nitrophenyl octyl ether (NPOE) for 5 seconds. The excess NPOE was gently removed with a medical wipe. A volume of 7 μl of 10 mM formic acid was filled with an airtight syringe in the lumen of each of the three porous hollow fibers. Platinum wires with a diameter of 0.2 mm (K. A Rasmussen, Hamar, Norway) were connected to the power supply and utilized as electrodes. The anode was placed in the sample compartment and the three cathodes were placed in the lumen of the three porous hollow fibers, one in each of the hollow fibers. A power supply ES 0300-0.45 from Delta Power Supplies (Delta Electronika, Zierikzee, The Netherlands) was operated at 100 V. The sample compartment was agitated at 3000 rpm during extraction for 10 min with an IKA MS 3 digital vortex mixer (Staufen, Germany). The extracts obtained by EME were diluted 1:1 with mobile phase A and 20 μl was injected into LC-MS.

### Liquid Chromatography-Mass Spectrometry (LC-MS)

The chromatographic separation was performed on a Biobasic-C8 reversed-phase column (50×1 mm) from Thermo Fisher Scientific (Waltham, Mass.) with average pore size of 300 Å and particle diameter of 5 μm. The chromatographic system consisted of a Shimadzu SIL-10ADvp auto injector, two Shimadzu LC-10ADvp gradient pumps, a Shimadzu DGU-14A degasser, a Shimadzu SCL-10Avp system controller, and a Shimadzu LCMS-2010A single-quadrupole MS detector (all from Shimadzu Scientific Instruments, Kyoto, Japan). Data acquisition and processing were carried out using Shimadzu LCMS Solution software Version 2.04-H3.

The compositions of the mobile phases were as follows—mobile phase A: 20 mM formic acid and methanol (95:5, v/v); mobile phase B: 20 mM formic acid and methanol (5:95, v/v). In some analyses, a gradient was run from 0% mobile phase B up to 15% mobile phase; after 12 minutes, the mobile phase B was kept constant at 100% for 10 minutes. Eventually the column was reconditioned with 0% mobile phase B for 5.1 minutes. In some analyses, a linear gradient was run from 20% mobile phase B up to 100% mobile phase B; after 15 minutes, the mobile phase composition was kept constant for 3 minutes. Eventually the column was reconditioned with 20% mobile phase B for 6.1 minutes. The injection volume was set to 20 μL and the mobile phase flow rate was 50 μL/min.

The MS was operated with an electro spray ionization (ESI) source operated in the positive ionization mode to interface the HPLC and the MS. Analyses were performed with selected ion monitoring (SIM). The m/z values of 468, 310, 150, 166, and 180 were used for buprenorphine, methadone, methamphetamine, para-methoxyamphetamine, and para-methoxymethamphetamine, respectively. The m/z values of 325, 341, 477, and 306 were used for citalopram, citalopram hydrobromide (Lu 10-202, internal standard), loperamide, and sertraline, respectively.

The MS operating conditions were as follows: flow rate of drying gas: 15 L/min; flow rate of nebulizing gas: 1.5 L/min; temperature of the curved desolvation line: 200° C.; block temperature: 200° C.; and probe voltage: +4.5 kV.

### Post-Column Infusion

For the oral fluid analysis, a standard solution of buprenorphine, methadone, methamphetamine, para-methoxyamphetamine, and para-methoxymethamphetamine with a concentration of 500 ng/ml in 0.1% TFA was infused directly with a syringe pump (Gilson 402 Dilutor dispenser, Middleton, Wis.) into the ESI source with a T-piece. For the blood spot analysis, a standard solution of citalopram, Lu 10-202, loperamide, methadone, and sertraline with a concentration of 200 ng/ml in 10 mM formic acid was infused directly with a syringe pump (Gilson 402 Dilutor dispenser, Middleton, Wis., USA) into the ESI source with a T-piece. The T-piece connected the mobile phase flow from the chromatographic separation with the standard solution delivered from the syringe pump. Eventually, 20 μL of an EME extract from a blank dried oral or blood fluid spot was injected onto the chromatographic column to examine potential matrix effects and/or ion suppression.

### Calculation of Extraction Recovery

Extraction recoveries (R) for the model substances were calculated according to the following equation:

$$R = \frac{{n}_{a , final}}{{n}_{s , initial}} \cdot 100 \% = ( \frac{{V}_{a}}{{V}_{s}} ) ( \frac{{C}_{a , final}}{{C}_{s , initial}} ) \cdot 100 \%$$

where n_{a,final} is the moles of analyte that is collected in the acceptor solution and the moles of the analyte initially present in the donor solution. Vₐ and Vₛ are the volumes of the acceptor solution and the sample solution, respectively. C_{a,final} is the final concentration of the analyte in the acceptor solution and C_{s,initial} is the concentration of the analyte in the donor solution prior to extraction.

### Example 1

### Chitosan and Alginate Foam as Sampling Medium for Oral Fluid

Conventional oral fluid sampling procedures are based on sampling with a pad in the oral cavity, then storing the sampling pad in a diluent prior to sample preparation and analysis. Problems with eluting the analytes of interest from the sampling pad increase the probability for false negative results, especially in the case of highly lipophilic drugs. The massive matrix effect due to constituents in the diluent is another major concern with conventional sampling procedures. Restricted oral fluid secretion, due to physiological conditions or drug use, is another issue, which can make the sampling procedure highly demanding.

During the development of embodiments of the technology to address these problems, experiments were performed to examine storage of low microliter volumes of oral fluid as dried drops on alginate and chitosan foams. A 10-μl volume of a freshly prepared oral fluid spiked with model analytes (buprenorphine, methadone, methamphetamine, para-methoxyamphetamine (PMA), and para-methoxymethamphetamine (PMMA)) was spotted on the biopolymer of interest, dried under room temperature for 2 hours, and subsequently punched out with a homemade puncher. A volume of 10 μl oral fluid occupied approximately a diameter of 5 mm on the biopolymer. To maintain volume control and reproducible punches, an 8-mm diameter circle was punched out, which included the entire oral fluid drop of 10 μl.

The punched out dried oral fluid sample was subsequently dissolved in 1 mM hydrochloric acid for 5 minutes with stirring at 3000 rpm. In the case of alginate this dissolution procedure provided a clear translucent solution of alginate, oral fluid, and model analytes. In the case of chitosan a small portion of undissolved matter was observed after 5 minutes of dissolution. Nevertheless, the solutions provided after a 5 minute of dissolution of alginate or chitosan were considered satisfactory for further processing.

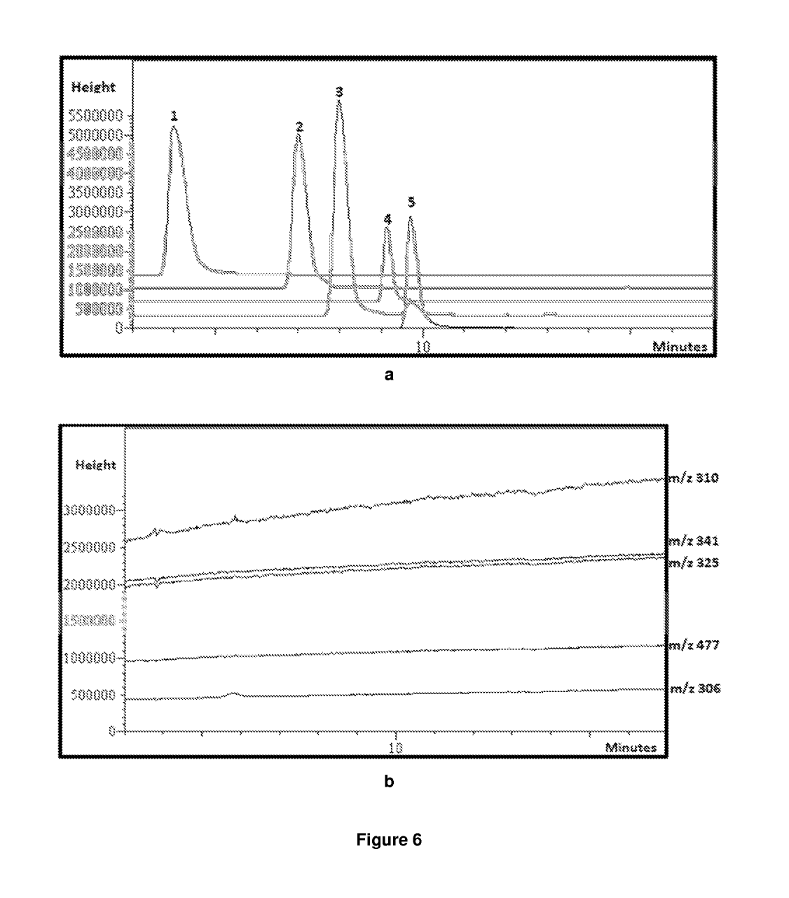

A selective extraction technique was used to isolate the analyte from the complex matrix comprising dissolved biopolymer, model analytes, and hydrochloric acid. The analytes were isolated electrokinetically by EME from the sample solution, through a SLM and into 0.1% TFA within 5 minutes of extraction. The EME extract was diluted 1:1 with mobile phase A and injected directly into the LC-MS. The combination of oral fluid sampling on alginate or chitosan, dissolution, and subsequent EME-LC-MS provided clean extracts and chromatograms with low baseline noise. A chromatogram from oral fluid spiked with model analytes and stored on alginate foam is shown in FIG. 6.

The proposed method has been evaluated with respect to the FDA guidance for industry concerning bioanalytical method validation. Sampling on both alginate and chitosan were validated, and the results are summarized in Tables 1 and 2.

TABLE 1
Validation results with alginate combined with EME-LC/MS
from dried oral fluid spiked with model analytes.

| | | Correla- | | | | |
|---|---|---|---|---|---|---|
| | Calibra- | tion | | | | Extrac- |
| | tion | coeffi- | | Concen- | | tion Re- |
| | range | cient | LLOQ | tration | RSD* | covery* |
| Analyte | (ng/ml) | R² | ng/ml | ng/ml | % | % |
| Buprenorphine | 25-1000 | 0.9905 | 13 | 50 | 10 | 25 |
| | | | | 1000 | 7 | 31 |
| Methadone | 25-1000 | 0.9922 | 2 | 50 | 11 | 55 |
| | | | | 1000 | 6 | 77 |
| Meth- | 25-1000 | 0.9987 | 18 | 50 | 7 | 62 |
| amphetamine | | | | 1000 | 3 | 82 |
| PMA | 25-1000 | 0.9936 | 10 | 50 | 11 | 51 |
| | | | | 1000 | 7 | 62 |
| PMMA | 50-1000 | 0.997 | 25 | 50 | 4 | 65 |
| | | | | 1000 | 7 | 74 |

*n = 6

In Table 1, the sample is 10 μl of a dried alginate oral fluid drop dissolved in 300 μl 1 mM HCl; the SLM is 10% TEHP in IPNB (w/w); the acceptor solution is 25 μl of 0.1% TFA; the extraction voltage is 25 V, and the extraction time is 5 minutes.

TABLE 2
Validation results with chitosan combined with EME-LC/MS
from dried oral fluid spiked with model analytes.

| | | Correla- | | | | |
|---|---|---|---|---|---|---|
| | Calibra- | tion | | | | Extrac- |
| | tion | coeffi- | | Concen- | | tion Re- |
| | range | cient | LOQ | tration | RSD* | covery* |
| Analyte | (ng/ml) | R² | ng/ml | ng/ml | % | % |
| Buprenorphine | 25-1000 | 0.9885 | 13 | 50 | 12 | 30 |
| | | | | 1000 | 6 | 42 |
| Methadone | 25-1000 | 0.9951 | 2 | 50 | 5 | 64 |
| | | | | 1000 | 8 | 69 |
| Meth- | 25-1000 | 0.9955 | 18 | 50 | 15 | 35 |
| amphetamine | | | | 1000 | 17 | 54 |
| PMA | 25-1000 | 0.9963 | 10 | 50 | 13 | 28 |
| | | | | 1000 | 16 | 48 |
| PMMA | 50-1000 | 0.9908 | 25 | 50 | 10 | 61 |
| | | | | 1000 | 14 | 64 |

*n = 6

In Table 2, the sample is 10 μl of dried oral fluid on chitosan dissolved in 300 μl 1 mM HCl; the SLM is 10% TEHP in IPNB (w/w); the acceptor solution is 25 μl of 0.1% TFA; the extraction voltage is 25 V; and the extraction time is 5 minutes.

Recovery was investigated with the EME setup described herein. Oral fluid samples at 3 different concentration levels were prepared in fresh oral fluid and spotted on alginate and chitosan. Recovery for all the 5 model analytes differs only slightly as shown in Table 1 and Table 2. Considering the homebuilt equipment, the viscous nature of oral fluid, and the small volumes that must be precisely applied (10 μl), the recovery was considered as independent of analyte concentration in the oral fluid.

Both the limit of detection (LOD) (defined as signal to noise ratio of 3) and the limit of quantification (LOQ) (defined as signal to noise ratio of 10) were examined with respect to buprenorphine, methadone, methamphetamine, para-methoxyamphetamine, and para-methoxymethamphetamine.

The combination of sensitive LC-MS analysis with the relatively high recoveries obtained with EME contributed to a LOQ close to the cut-off levels defined by DRUID, although only 10 μl of oral fluid was applied to the sampling medium for each analysis. EME from whole blood for some similar analytes by utilizing UPLC-MS-MS reported LOD and LOQ well below the cut off levels defined by DRUID (Eskandari, supra).

Linearity was examined for both alginate and chitosan foam as sampling medium in the range 25-1000 ng/ml. A level of 25 ng/ml is close to the DRUID cut-off levels for most of the model analytes. The reported correlation coefficients (r²) with alginate as storage medium were 0.9905, 0.9922, 0.9987, 0.9936, and 0.997 in the case of buprenorphine, methadone, methamphetamine, para-methoxyamphetamine, and para-methoxymethamphetamine, respectively. The reported correlation coefficients (r²) with chitosan as storage medium were 0.9885, 0.9951, 0.9955, 0.9963, and 0.9908 in the case of buprenorphine, methadone, methamphetamine, para-methoxyamphetamine, and para-methoxymethamphetamine, respectively.

The correlation coefficients were considered as satisfactorily for both alginate and chitosan with regard to the FDA guidance for industry concerning bioanalytical method validation.

The relative standard deviation (RSD %) was investigated at 50- and 1000 ng/ml for alginate and chitosan foam. The reported RSD values for low concentration, defined as 50 ng/ml, were below 15% for all model analytes on both alginate and chitosan foam. High concentration level, defined as 1000 ng/ml, reported RSD values below 20%.

The RSD values for low and high concentrations of model analytes by utilizing alginate and chitosan as sampling medium were considered satisfactory with regard to the FDA guidance for industry concerning bioanalytical method validation.

To investigate the long term stability of the model analytes, freshly prepared oral fluid samples with a concentration of 100 and 500 ng/ml were spotted on alginate and chitosan foam and dried at room temperature for 3 hours. Subsequently, the dried oral fluid was punched out and stored in air tight bags for 30 days at the following conditions: room temperature, 37° C., −18° C., and 4° C. At day 30, the punched out dried oral fluid sample was dissolved in 300 μl 1 mM HCl, extracted with the described EME setup, and analyzed with LC-MS. The results are shown in Tables 3 and 4 for alginate and chitosan foam, respectively. No sample loss is observed after 30 days of storage. The stability of the model analytes by storing oral fluid on alginate and chitosan foam was considered satisfactory.

TABLE 3
Measured concentration (ng/ml) at day 30 after
spotting oral fluid or alginate foam with a concentration
of 100 and 500 ng/ml, respectively.

| Analyte | Theoretical | 37 C. ° | −18 C. ° | 4 C. ° | RT * |
|---|---|---|---|---|---|
| Buprenorphine | 100 | 115 | 96 | 101 | 97 |
| | 500 | 656 | 656 | 790 | 642 |
| Methadone | 100 | 117 | 106 | 113 | 103 |
| | 500 | [symbol] 81 | 602 | 636 | 582 |
| Methamphetamine | 100 | 42 | 103 | 107 | 72 |
| | 500 | 319 | 687 | 645 | 466 |
| PMA | 100 | 53 | 105 | 115 | 80 |
| | 500 | 406 | 717 | 722 | 628 |
| PMMA | 100 | 76 | 114 | 114 | 197 |
| | 500 | 475 | 726 | 715 | 628 |

n = 3;
RT signifies room temperature [symbol]
indicates data missing or illegible when filed

In Table 3, the sample is 10 μl dried oral fluid on alginate dissolved in 300 μl 1 mM HCl; SLM is 10% TEHP in IPNB (w/w); acceptor solution is 25 μl of 0.1% TFA; the extraction voltage is 25 V; and the Extraction time is 5 minutes.

TABLE 4
Measured concentration (ng/ml) under different storage conditions
at day 30 after spotting oral fluid on chitosan foam with
a concentration of 100 and 500 ng/ml, respectively.

| Analyte | Theoretical | 37 C. ° | −18 C. ° | 4 C. ° | RT * |
|---|---|---|---|---|---|
| Buprenorphine | 100 | 100 | 101 | 105 | 101 |
| | 500 | 595 | 611 | 569 | 595 |
| Methadone | 100 | 108 | 105 | 115 | 113 |
| | 500 | 545 | 599 | 564 | 582 |
| Methamphetamine | 100 | 80 | 117 | 126 | 98 |
| | 500 | 467 | 732 | 744 | 601 |
| PMA | 100 | 131 | 122 | 127 | 104 |
| | 500 | 648 | 769 | 765 | 719 |
| PMMA | 100 | 112 | 119 | 123 | 104 |
| | 500 | 623 | 769 | 765 | 647 |

n = 3;
RT signifies room temperature

In Table 4, the sample is 10 μl dried oral fluid on chitosan dissolved in 300 μl 1 mM HCl; SLM is 10% TEHP in IPNB (w/w); acceptor solution is 25 μl of 0.1% TFA; the extraction voltage is 25 V; and the extraction time is 5 minutes.

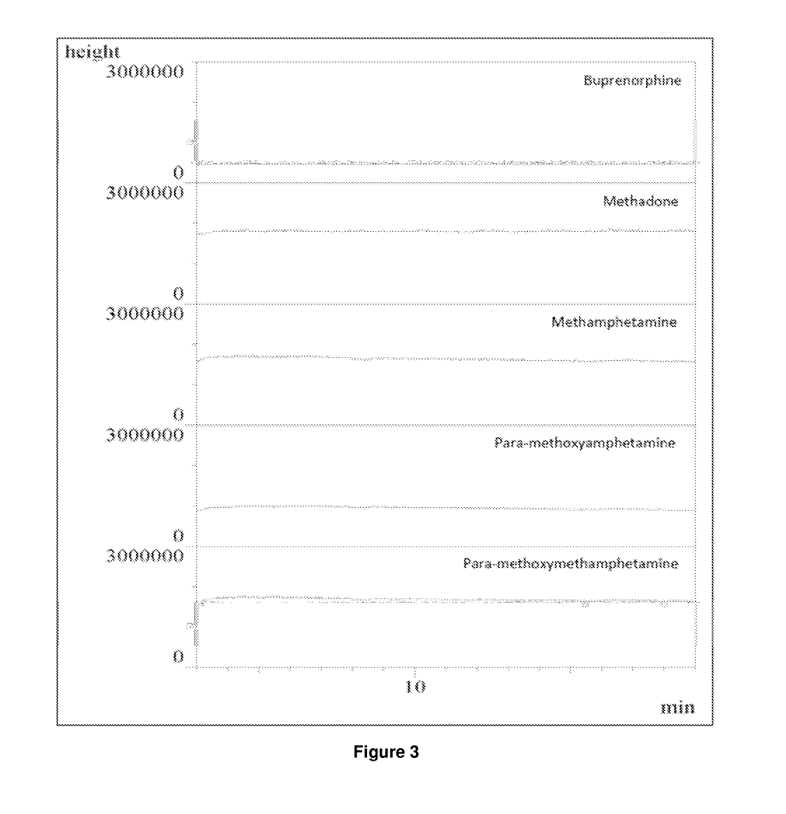

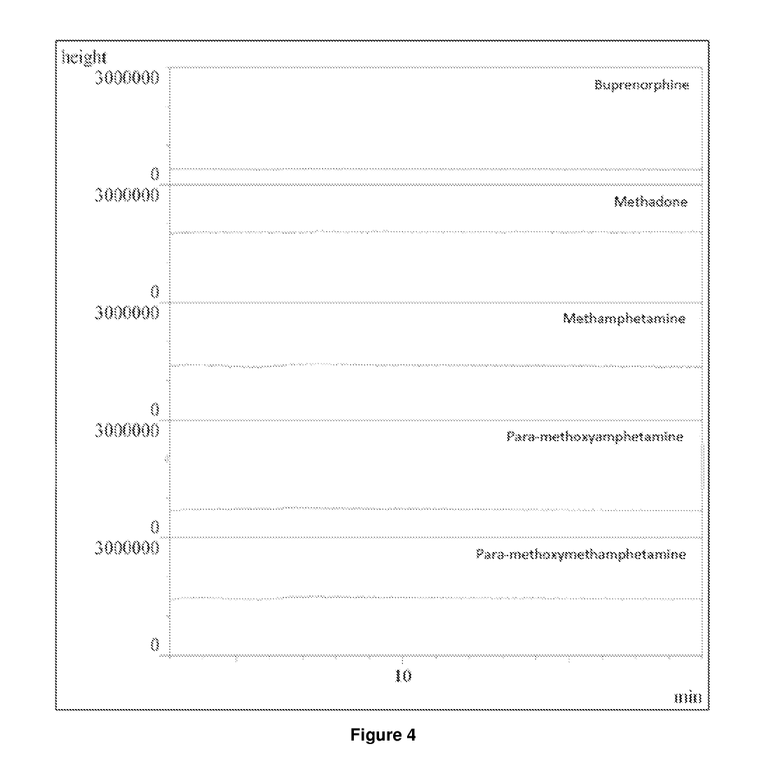

Co-extraction of matrix components from the sample and into the final extract is reported to be a major concern in terms of LC-MS analysis, especially regarding analysis conducted from oral fluid. To investigate potential matrix effect with the proposed method, a post-column infusion experiment was conducted as described herein. The post-column chromatograms obtained are shown in FIGS. 3 and 4 for alginate and chitosan foam, respectively. Neither signal enhancement nor suppression in the relevant retention time interval was observed. To summarize, there is no indication of matrix effect in the final extract obtained with the proposed method and FIGS. 3 and 4 show data that EME provides excellent sample cleanup.

### Example 2

### Chitosan and Alginate Foam as Sampling Medium for Blood

During the development of embodiments of the technology provided, experiments were performed to assess the time needed to dissolve a punched out DBS. A 10-μl volume of whole blood samples containing 280 ng/mL of citalopram, methadone, loperamide, and sertraline as model substances was accurately spotted with a micro syringe. After drying for 2 hours at room temperature, the whole blood spot was punched out and dissolved in various acidic solutions. A DBS spotted on alginate foam was completely dissolved after 3 min in 1 mM HCl. This solution had a pH of 3, which is in the lower part of the solubility range for dissolution of alginate. A punched out DBS dissolved in 300 μl of 1 mM HCl produces a translucent reddish solution, indicating the complete dissolution of the polymer and the DBS. Chitosan dissolved more slowly and after 5 minutes a small portion of undissolved matter was still observed in the bottom of the tube. The small portion of undissolved matter in the chitosan solution did not reduce recoveries. Prior to the experiment there was concern about possible ionic interactions between the basic analytes and negatively charged groups on alginate, which could lead to reduced recoveries. Because the recoveries from alginate and chitosan were similar and close to 100%, there were no indications of such interactions at the conditions used.

In this investigation, EME was selected because of the speed of extraction. In the three-fiber setup, extraction times were 10 min and the analytes were collected in 21 μl of 10 mM formic acid. The extract was diluted 1:1 with mobile phase A prior to injection of 20 μl into LC-MS. The recoveries obtained after extraction of the model analytes from solutions of DBS on alginate and chitosan are shown in Table 5.

TABLE 5
Recoveries obtained with EME by utilizing alginate and
chitosan as storage medium for dried blood spots, n = 3.

Extraction recovery, (RSD, %)

| | Alginate | Chitosan |
|---|---|---|
| Citalopram | 100% (4) | 103% |
| Methadone | 105% (6) | 115% |
| Loperamide | 90% (6) | 91% |
| Sertraline | 44% (4) | 57% |

In Table 5, the sample is 10 μl dried alginate blood drop dissolved in 300 μl 1 mM HCl; SLM is NPOE; acceptor solution is 21 μl of 10 mM HCOOH; extraction voltage is 100 V; and extraction time is 10 minutes.

The recoveries were in the range of 44-115% and were similar to the ones reported for extraction of the model analytes from wet plasma (Eibak, supra). These data show that the presence of the dissolved biopolymer and blood components do not interfere with the extractions.

Elution of analytes from DBS on conventional cards was performed using the standard procedures recommended by Agilent and Whatman. The recoveries obtained by using the standard procedures are shown in Table 6.

TABLE 6
Recovery for model substances by utilizing Agilent
Bond Elute DMS and Whatman PTA DMPK-A, n = 3.

Extraction recovery (RSD, %)

| | Bond Elute DMS | FTA DMPK-A |
|---|---|---|
| Citalopran | 69% (6) | 17% (17) |
| Methadone | 59% (7) | 33% (12) |
| Loperamide | 59% (4) | 24% (8) |
| Sertraline | 66% (5) | 11% (3) |

Elution of analytes from Agilent Bond Elute DMS with the procedure recommended by Agilent produced recoveries in the range 59-69%. This procedure involved elution for 1 hour, centrifugation, evaporation to dryness, and reconstitution in the mobile phase. The recoveries from Whatman FTA DMPK-A cards using elution for 10 minutes were considerably lower (11-33%), most probably because the recommended elution time of 10 minutes is far too short for elution of the model substances studied in this investigation.

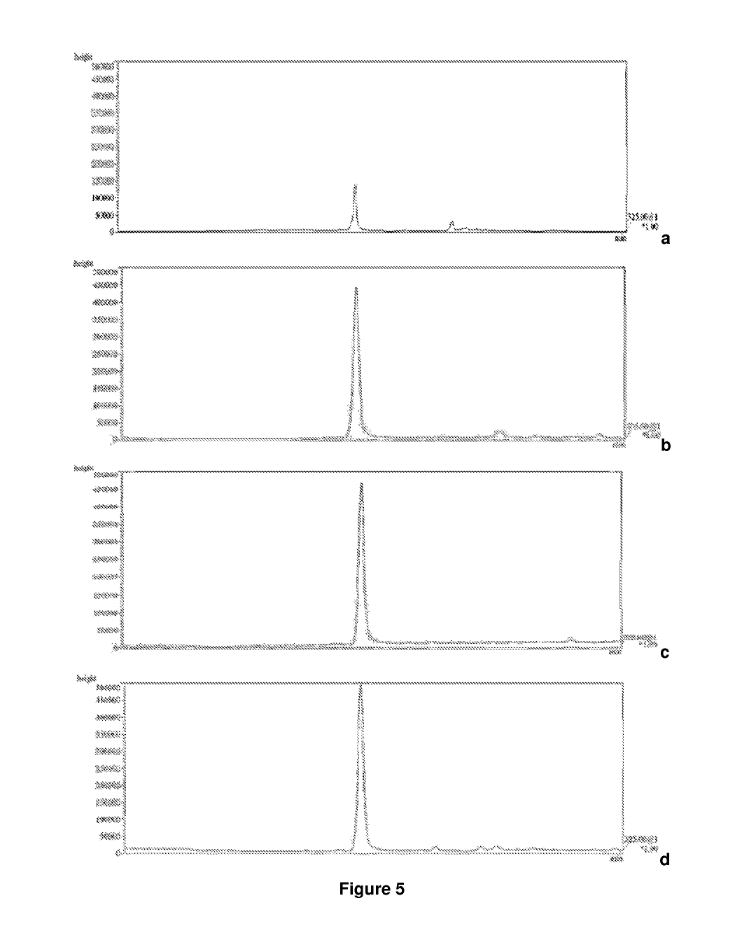

Sufficient recoveries are obtained with the procedure used for Bond Elute DMS but the time needed is significantly higher compared the procedure used for biopolymers. The use of biopolymers as a sampling medium for dried blood spots can therefore offer a considerable saving of time. FIG. 5 shows chromatograms of citalopram from DBS on FTA DMPK-A (a), Bond Elute DMS (b), alginate-EME (c), and alginate followed by precipitation with ice-cold acetonitrile (d). Small addition peaks are seen as baseline noise in the chromatograms. In the chromatogram from alginate-EME the baseline noise is almost eliminated by the extraction. The liquid membrane utilized in the case of alginate-EME acts as a selective cleanup barrier and excludes larger proteins and reduces the co-extraction of impurities. This experiment demonstrates that EME provides clean extracts from complex biological matrices that are directly compatible with LC-MS. The time needed for sample preparation is 15 minutes including dissolution and extraction.

The proposed method has been evaluated with respect to the FDA guidance for industry concerning bioanalytical method validation (Table 7).

TABLE 7
Validation results with alginate combined with EME-LC/MS
from dried blood spot spiked with model substances.

| | | | Meth- | |
|---|---|---|---|---|
| | Citalopram | Loperamide | adone | Sertaline |
| LOD (ng/ml) | 0.4 | 1.6 | 0.6 | 5.3 |
| LOQ (ng/ml) | 1.2 | 5.5 | 2.0 | 5.3 |
| | Linearity | | | |
| Range (ng/ml) | 17.5-560 | 17.5-560 | 17.5-560 | 17.5-560 |
| R² | 0.983 | 0.986 | 0.992 | 0.9952 |
| | Repeatability (%) [symbol] | | | |
| 1120 ng/ml (n = 5) | 4 | 6 | 6 | 4 |
| 140 ng/ml (n = 5) | 17 | 15 | 14 | 12 |
| 17.5 ng/ml (n = 5) | 5 | 20 | 16 | 18 |

[symbol]

indicates data missing or illegible when filed

In Table 7, the sample is 10 μl dried alginate blood drop dissolved in 300 μl 1 mM HCl; SLM is NPOE; acceptor solution is 21 μl of 10 mM HCOOH; extraction voltage is 100 V; and extraction time is 10 minutes. ^{a}Lu 10-202 used as internal standard for citalopram, loperamide, methadone, and sertraline.

Both the limit of detection (LOD) (defined as signal to noise ratio of 3) and the limit of quantification (LOQ) (defined as signal to noise ratio of 10) were examined with respect to citalopram, loperamide, methadone, and sertraline. The LOQ was below the lower therapeutic level for all model substances (Schulz M, Schmoldt A. Therapeutic and toxic blood concentrations of more than 800 drugs and other xenobiotics. Pharmazie 2003; 58:447-74). The combination of sensitive LC-MS analysis with the high recoveries obtained with EME contributed to LOQ below the normal therapeutic range, in spite of that only 10 μl whole blood was used for each analysis.

Linearity was investigated in the range 17.5 ng/m1-560 ng/ml; this interval included the therapeutic range for citalopram, loperamide, methadone, and sertraline. As stated in Table 7, the correlation coefficients (r²) were 0.9986, 0.992, and 0.9952 in the case of loperamide, methadone, and sertraline, respectively. The reported correlation coefficient regarding citalopram was 0.983, and thus below the FDA guidance for industry concerning bioanalytical method validation. Considering the homebuilt equipment and the small blood volumes that must be accurately applied (10 μl), the linearity was considered as satisfactory.

To establish the relative standard deviation (RSD %), repeatability was examined for low (17.5 ng/ml), medium (140 ng/ml), and high concentration (1120 ng/ml) of citalopram, methadone, loperamide, and sertraline, respectively. As stated in Table 7, the RSD is below 20% for all the four model substances regardless of the whole blood concentration. The RSD values reported in Table 7 are thus satisfactory with regard to the FDA guidance for industry concerning bioanalytical method validation.

To compare the absolute recovery of citalopram, methadone, loperamide, and sertraline for three different DBS mediums, four whole blood samples were spotted on each medium and extracted with the described EME setup. Alginate foam provided recoveries close to 100% for three of the model substances. The recoveries obtained are comparable to the ones obtained for the same substances from wet plasma in an identical EME setup (Eibak, supra).

Potential ion suppression of the signal in the MS due to co-extracted matrix components is a major concern regarding LC-MS analysis. Ion suppression was thus examined by post-column infusion of the model substances as outlined herein.

The post column infusion chromatograms are shown in FIG. 6a. A chromatogram showing retention times for the model substances and internal standards is shown in FIG. 6b. There were no indications of ion suppression, thus supporting earlier findings that EME provides excellent sample cleanup.

To investigate the stability of the model substances, freshly prepared whole blood samples with a concentration of 70 ng/ml were spotted on alginate foam and dried for 3 hours at room temperature. Subsequently, the DBS were stored in air tight bags for 50 days at room temperature, 37° C., −18° C., and 4° C. At day 50, the DBS were dissolved, extracted with the described EME apparatus, and analyzed with LC-MS. The results are shown in Table 8.

TABLE 8
Nominal concentration (ng/ml) at day 50 after spotting whole
blood with citalopram, methadone, loperamide, and sertraline
with a concentration of 70 ng/ml on alginate foam.
Nominal concentration (ng/ml)

Storage condition

| Model substance | 37° C. | −18° C. | 4° C. | RT* |
|---|---|---|---|---|
| Citalopram | 59 | 72 | 64 | 64 |
| Methadone | 54 | 79 | 63 | 61 |
| Loperamide | 59 | 70 | 58 | 53 |
| Sertraline | 61 | 64 | 69 | 77 |

In Table 8, the sample is 10 μl dried alginate blood drop dissolved in 300 μl 1 mM HCl; SLM is NPOE, acceptor solution is 21 μl of 10 mM HCOOH; the extraction voltage is 100 V; and the extraction time is 10 minutes. *Room temperature

No sample loss is observed after storage at −18° C. To summarize, the stability of the model substances by utilizing alginate foam as storage medium for the dried blood drop was considered as satisfactory.

### Example 3

### Extraction Recoveries from CMC

During the development of embodiments of the technology provided herein, experiments were performed to determine the recovery of model analytes from carboxymethylcellulose. The data collected are provided in Table 9.

TABLE 9
Extraction recoveries from CMC

| | Extraction recovery |
|---|---|
| Citalopram | 107% |
| Methadone | 109% |
| Loperamide | 82% |
| Sertraline | 43% |

In Table 9, the sample is 10 μl of dried blood spotted on CMC, then dissolved in 300 μl of 1 mM HCl; SLM is NPOE; the acceptor solution is 21 μl of 10 mM HCOOH; the extraction voltage is 100 V; and the extraction time is 10 minutes.

### Example 4

### Extraction of Model Analytes from CMC/Ag, CMC, Alginate, and Cellulose Ethyl Sulphonate

During the development of embodiments of the technology provided herein, experiments were performed to evaluate the recovery of model analytes dissolved in body fluids and spotted on a range of sampling materials (e.g., a biopolymer).

The model analytes tested were pethidine, nortriptyline, methadone, haloperidol, and loperamide. These model analytes were dissolved in blood, plasma, oral fluid, or urine, and then spotted on a sampling material. Data were collected (Tables 10-13) for the extraction of analytes from body fluids spotted on carboxy methyl cellulose (e.g., AQUACEL, ConvaTec, Skillman, N.J.; Tables 10-13, “CMC”); carboxy methyl cellulose impregnated with silver (e.g., AQUACEL Ag, ConvaTec; Tables 10-13, “CMC/Ag”); cellulose ethyl sulphonate (e.g., DURAFIBER, Smith & Nephew, Melbourne, AU; Tables 10-13, “CES”); and two alginates (Alginat 3M, 3M, Saint Paul, Minn.; and Seasorb, Coloplast, Minneapolis, Minn.; Tables 10-13, “Alginate 1” and “Alginate 2”, respectively) as the sampling materials. The general procedures for extraction and isolation of analytes, LC-MS, and data collection were performed as described herein above and samples were dissolved in 300 μl of 1 mM HCl, the SLM was NPOE, the acceptor solution volume was 21 μl of 10 mM HCOOH, the extraction voltage was 100 V, and the extraction time was 10 minutes.

TABLE 10
recoveries of analytes from dried blood spots

| | CMC/Ag | CMC | Alginate 1 | CES | Alginate 2 |
|---|---|---|---|---|---|
| Pethidine | 59% | 58% | 53% | 79% | 74% |
| Nortriptyline | 70% | 68% | 80% | 82% | 78% |
| Methadone | 64% | 69% | 78% | 83% | 73% |
| Haloperidol | 29% | 30% | 33% | 33% | 32% |
| Loperamide | 66% | 66% | 68% | 68% | 67% |

TABLE 11
recoveries of analytes from dried plasma spots

| | CMC/Ag | CMC | Alginate 1 | CES | Alginate 2 |
|---|---|---|---|---|---|
| Pethidine | 70% | 71% | 81% | 84% | 54% |
| Nortriptyline | 75% | 76% | 80% | 86% | 63% |
| Methadone | 62% | 77% | 73% | 84% | 59% |
| Haloperidol | 30% | 33% | 34% | 36% | 26% |
| Loperamide | 61% | 70% | 64% | 74% | 55% |

TABLE 12
recoveries of analytes from dried oral fluid spots

| | CMC/Ag | CMC | Alginate 1 | CES | Alginate 2 |
|---|---|---|---|---|---|
| Pethidine | 90% | 77% | 73% | 66% | 81% |
| Nortriptyline | 70% | 68% | 106% | 64% | 80% |
| Methadone | 66% | 67% | 63% | 67% | 79% |
| Haloperidol | 32% | 30% | 30% | 30% | 36% |
| Loperamide | 65% | 63% | 54% | 61% | 76% |

TABLE 13
recoveries of analytes from dried urine spots

| | CMC/Ag | CMC | Alginate 1 | CES | Alginate 2 |
|---|---|---|---|---|---|
| Pethidine | 73% | 58% | 52% | 73% | 70% |
| Nortriptyline | 107% | 90% | 88% | 78% | 82% |
| Methadone | 58% | 36% | 47% | 63% | 60% |
| Haloperidol | 28% | 17% | 23% | 28% | 31% |
| Loperamide | 56% | 34% | 46% | 55% | 62% |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1. An article comprising a biopolymer spotting card and a biological fluid.

2. The article of claim 1 wherein the biopolymer is a sugar-based biopolymer.

3. The article of claim 1 wherein the biopolymer comprises water soluble cellulose derivatives, chitin, dextran, pullulan, hyaluronic acid and salts thereof, pectins, carrageenans, and/or xanthan.

4. The article of claim 1 wherein the biological fluid is blood, a blood fraction, or a component of blood.

5. The article of claim 1 wherein the biological fluid is oral fluid.

6. The article of claim 1 wherein the biological fluid is sputum, semen, sweat, urine, cerebrospinal fluid, tears, saliva, breast milk, or vaginal fluid.

7. The article claim 1 further comprising a dilute acid.

8. The article of claim 1 wherein the biological fluid comprises an analyte.

9. The article of claim 8 wherein the analyte is a drug, toxin, metabolite, nucleic acid, protein, lipid, or therapeutic agent.

10. The article of claim 1 further comprising a dye that is a visible color when contacted with the biological fluid.

11. The article of claim 1 wherein the biopolymer is formulated in the form of a porous sheet of foam.

12-13. (canceled)

14. A method for sampling a biological fluid, the method comprising:
a) spotting the biological fluid on a biopolymer spotting card; and
b) drying the biological fluid on the biopolymer spotting card.

15. The method of claim 14 further comprising storing the biological fluid on the biopolymer spotting card.

16. The method of claim 15 wherein the storing is for a period of time less than or equal to 50 days at room temperature.

17. A method of measuring an analyte, the method comprising:
a) spotting a biological fluid on a biopolymer spotting card;
b) dissolving the biopolymer spotting card;
c) recovering the analyte; and
d) measuring a property of the analyte.

18. The method of claim 17 further comprising cutting the biopolymer spotting card.

19. The method of claim 17 wherein the measuring comprises using mass spectrometry, using liquid chromatography, using gas chromatography, detecting fluorescence, detecting an ultraviolet signal, electrochemical detection, nitrogen-phosphor detection, immunological detection, free induction decay, and/or combinations thereof.

20. The method of claim 17 wherein the recovering comprises using an extraction technique.

21. The method of claim 20 wherein the recovering comprises using a technique selected from the group comprising electromembrane extraction, precipitation of the biopolymer, liquid-liquid extraction, solid-phase extraction, and combinations thereof.

22. The method of claim 17 wherein the dissolving comprises using a dilute acid.

23. The method of claim 22 wherein the dilute acid is hydrochloric acid.

24. The method of claim 17 further comprising storing the biological fluid on the biopolymer spotting card.

25-42. (canceled)
